(19) United States
(12) Patent Application Publication
(10) Pub. No.: US 2013/0011374 A1
(43) Pub. Date: Jan. 10, 2013

(54) GROWTH INHIBITION OF MICROORGANISMS BY LACTIC ACID BACTERIA

(75) Inventors: Francios-Marie Luquet, Orsay (FR); Mathieu Millette, Quebec (CA); Monique Lacroix, Quebec (CA)

(73) Assignee: Bio-K Plus International Inc.

(21) Appl. No.: 12/520,210

(22) PCT Filed: May 30, 2007
(86) PCT No.: PCT/CA2007/000962

§ 371 Date: Sep. 2, 2010

Related U.S. Application Data
(60) Provisional application No. 60/876,461, filed on Dec. 22, 2006.

(51) Int. Cl. A61K 35/74; A61P 1/00; A01P 1/00; A23C 21/02; A01N 63/02

(52) U.S. Cl. 424/93.45; 426/583; 424/535

(57) ABSTRACT

The present invention relates to growth inhibition of microorganisms by lactic acid bacteria. The present invention also relates to the reduction and/or treatment of food-borne pathogen infections and/or nosocomial infections. The present invention also relates to the inhibition of spoilage microorganisms in food products. The present invention further relates to the modulation of gut flora. The invention is also directed to the inhibitory potential of a whey obtained from lactic acid bacteria.

### FIELD OF INVENTION

The present invention relates to the field of growth inhibition of microorganisms by lactic acid bacteria.

### BACKGROUND OF THE INVENTION

Lactic acid bacteria (LAB) and gram-positive rod of cocci bacteria that have been widely used in various fermented food products around the world for many centuries. The preservative role and health benefits of fermented milk (Scheinbach, 1998), kefir (Adolfsson et al., 2004), and yogurt (Perdigon et al., 2003) are now recognized.

Some lactic acid bacteria are also referred to as probiotics. According to the currently approved definition by the World Health Organization, the term “probiotics” describes live microorganisms which confer an health benefit to an host.

Antimicrobial activities of LAB have been demonstrated in various species (Rossland et al., 2003; Ghrairi et al., 2004; Nes et al., 2004). Some LAB can prevent the adherence, establishment, invasion or toxin production of intestinal and/or vaginal pathogens (Gusils et al., 2003). Some LAB can also inhibit some pathogens by a pH reduction through production of organic acid such as acetic, propionic and/or lactic acid (Naaber et al., 2004) or by producing hydrogen peroxide. LAB can also compete for nutrients or adhesion sites against pathogens. Furthermore, LAB can secrete antimicrobial peptides ribosomally synthesized named bacteriocins (nisin, pediocin, acidocin, etc). Bacteriocins from LAB are low molecular weight, cationic, amphiphilic molecules that are secreted by both Gram-positive as well as Gram-negative bacteria. Finally, antimicrobial activities of LAB may be related to their production of diacetyls compounds.

Microbial infections may occur by diverse routes of transmission. A common infection route relates to the ingestion of contaminated food, potentially leading to food-borne diseases. Some food-borne diseases can also be caused by toxins secreted by bacteria such as the toxin secreted by Staphylococcus aureus that may cause intense vomiting.

All individuals are at risk of contracting food-borne diseases with an increased preponderance in the very young, elderly, and the immunocompromised individuals. Food-borne diseases create important social and economic burdens and are a serious public health problem. Moreover, changes in human demographics, food preferences, food manufacturing and distribution, microbial adaptation and in public health systems have led to the emergence and reemergence of food-borne diseases.

Interestingly, according to De Buyser et al., milk and its derivatives are responsible for 1 to 5% of the total food-borne diseases outbreaks in seven industrialized countries. Salmonella spp., Staphylococcus aureus, Escherichia coli and Listeria monocytogenes are believed to be the main etiologic agents of these outbreaks.

Another increasingly common transmission path for pathogens relates to infections acquired in healthcare settings (nosocomial infections). The prevalence of such infections is on the rise.

There is a clear need for new agents to control microorganisms either by reducing or inhibiting their growth. Lactic acid bacteria (probiotics) may be used as biotherapeutic agents and help in resolving public health issues such as food-borne diseases and nosocomial infections.

The present invention seeks to meet these and other needs.

The present description refers to a number of documents, the content of which is herein incorporated by reference in their entirety.

### SUMMARY OF THE INVENTION

The present invention relates to the use of at least one lactic acid bacterium strain for inhibiting (reducing, decreasing, lowering, impairing) the growth of microorganisms.

Exemplary microorganisms of the present invention may be food-borne pathogens, nosocomial pathogens and/or spoilage microorganisms.

The present invention also relates to the use of at least one lactic acid bacterium strain for reducing and/or treating food-borne pathogen infections; for reducing or treating nosocomial infections and/or for inhibiting (reducing, decreasing, lowering, impairing) spoilage microorganisms in food products.

The present invention further relates to a whey obtained from fermentation using at least one lactic acid bacterium strain, its uses and methods of use.

The present invention is also directed to a method for inhibiting the growth of microorganisms, for reducing and/or treating food-borne pathogen infections as well as reducing and/or treating nosocomial infections which may comprise the step of administering an effective amount of at least one lactic acid bacterium strain and/or of a whey obtained from fermentation using at least one lactic acid bacterium strain.

The present invention further relates to a method to inhibit spoilage microorganisms which may comprise the step of adding an effective amount of at least one lactic acid bacterium strain to a food product and/or adding an effective amount of a whey obtained from at least one lactic acid bacterium strain.

The present invention is also directed to the use of at least one lactic acid bacterium strain or a whey obtained from fermentation using at least one lactic acid bacterium strain for modulating the gut flora and a method for modulating the gut flora which may comprise the step of administering an effective amount of at least one lactic acid bacterium strain and/or a whey obtained from fermentation using at least one lactic acid bacterium strain.

Finally, the present invention also relates to a food product which may comprise a whey.

### BRIEF DESCRIPTION OF THE DRAWINGS

In drawings which illustrates non-limitative exemplary embodiments of the present invention,

FIG. 1 illustrates the inhibition of pathogenic bacteria in associative culture (co-culture) with probiotic CL1285 in skimmed milk at 37° C. (□) LAB concentration during associative culture; () Pathogenic bacteria concentration during associative culture; (⋄) Pathogenic bacteria concentration during mono-culture. (A) Escherichia coli ATCC 25922; (B) E. coli O157:H7 EDL933; (C) Listeria innocua LSPQ 3285; (D) Staphylococcus aureus ATCC 25923; (E) Enterococcus faecalis LSPQ 2724; (F) E. faecium LSPQ 3550; (G) Salmonella Typhimurium SL1344;

FIG. 2 illustrates lactic acid bacteria population in feces of C57BI/6 mice during feeding with probiotic bacteria CL1285;

FIG. 3 illustrates Lactobacilli population in feces of C57BI/6 mice during feeding with probiotic bacteria CL1285;

FIG. 4 illustrates Enterobacteriaceae population in feces of C57BI/6 mice during feeding with probiotic bacteria CL1285;

FIG. 5 illustrates Staphylococci population in feces of C57BI/6 mice during feeding with probiotic bacteria CL1285; and

FIG. 6 illustrates the content of the total mesophilic anaerobes population in feces of C57BI/6 mice during feeding with probiotic bacteria CL1285.

### DETAILED DESCRIPTION OF THE INVENTION

In order to provide a clear and consistent understanding of the terms used in the present disclosure, a number of definitions are provided below. Moreover, unless defined otherwise, all technical and scientific terms as used herein have the same meaning as commonly understood to one of ordinary skill in the art to which this invention pertains.

As used in the specification and claim(s), the words ‘comprising’ (and any for of comprising, such as ‘comprise’ and ‘comprises’), ‘having’ (and any form of having, such as ‘have’ and ‘has’), ‘including’ (and any form of including, such as ‘include’ and ‘includes’) or ‘containing’ (and any form of containing, such as ‘contain’ and ‘contains’), are inclusive or open-ended and do not exclude additional, unrecited elements.

The present invention relates in one aspect thereof, to the use of at least one lactic acid bacterium strain (and/or a whey obtained from fermentation using at least one lactic acid bacterium) for inhibiting (reducing, decreasing, lowering, impairing) the growth of microorganisms. Inhibition may be partial and/or complete.

Such inhibition may occur at any time following contact of a lactic acid bacterium strain with microorganisms. For example, inhibition may occur from 0.1 to 48 h after contact. The present invention relates to, and explicitly incorporates herein, each and every specific member and combination of contact time sub-ranges whatsoever. Inhibition of growth may increase the generation time of a microorganism. Such increase in generation time may be from 101% up to 1000%. The present invention relates to, and explicitly incorporates herein, each and every specific member and combination of increase in generation time sub-ranges whatsoever.

In accordance with an embodiment of the present invention, the lactic acid bacterium strain may be resistant to bile salts. For example, the bacterium strain may resist to a concentration of bile salts of up to about 50 mg L-1. Furthermore, the lactic acid bacterium strain may be resistant to acidic environments (acid resistant). For example, the lactic acid bacterium strain may resist to a pH range of between 2 to 7 (from 2 to 7, from 2 to 3, from 2.5 to 3.5, from 2.5 to 3, etc). The present invention relates to and explicitly incorporates herein each and every specific member and combination of pH sub-ranges whatsoever. By ‘resist’ (resistance, resistant), it is meant that the growth of the lactic acid bacterium strain may not be completely inhibited.

In the present invention, the lactic acid bacterium strain may be of the genus Lactobacillus. In an exemplary embodiment of the present invention, the lactic acid bacterium strain may be Lactobacillus acidophilus, Lactobacillus casei and/or mixture thereof (such as, for example, the CL1285 mixture).

By ‘mixture’ it is meant the combination of lactic acid bacterium strains in any given proportions. The mixture of the present invention may comprise L. acidophilus I-1492 strain. For example such mixture may comprise about 95% of L. acidophilus strain I-1492 and/or about 5% of L. casei.

Any strains of Lactobacillus acidophilus or Lactobacillus casei may be used as long as they do not show deleterious effect. These strains may be of commercial origin and may be purchased from manufacturers of lactic ferments. In an exemplary embodiment, the Lactobacillus acidophilus strain may comprise strain I-1492 deposited on Nov. 15, 1994 at the Collection Nationale de Cultures de Microorganismes (CNCM; Institut Pasteur, 28 Rue du Docteur Roux, F-75724, Paris, CEDEX 15) according to the provisions of the Budapest Treaty.

Microorganisms' of the present invention may be food-borne pathogens, nosocomial pathogens and/or spoilage microorganisms. By “pathogen” it is meant a microorganism that may elicit a disease response in an individual.

Food-borne pathogens' are pathogens that may grow in food products and/or may enter the body following ingestion of food. Food-borne pathogens may also enter the body via other infection routes (eg. cutaneous, pulmonary, reproductive, etc.). Food borne pathogens may be, without limitation, Escherichia coli, Escherichia coli serotype O157:H7, Staphylococcus aureus, Listeria innocua, Enterococcus faecium, Enterococcus faecalis, Listeria monocytogenes and Salmolla Typhimurium.

Nosocomial pathogens' are pathogens present in the hospital, clinic and/or geriatric settings that may infect a visiting patient as a result of hospitalization or treatment. Such infection may be due to poor hygiene conditions in the hospital, clinic and/or geriatric settings. Exemplary nosocomial pathogens of the present invention may be selected from the group consisting of, but not limited to, Escherichia coli, Escherichia coli serotype O157:H7, Staphylococcus aureus, Listeria innocua, Enterococcus faecium, Enterococcus faecalis, Listeria monocytogenes and Salmolla Typhimirium. The nosocomial pathogens of the present invention may be selected from the group of Enterococcus faecium, Enterococcus faecalis, Escherichia coli and/or Listeria innocua.

Microorganisms of the present invention may be gram-positive bacteria and/or gram-negative bacteria. Exemplary gram-positive bacteria of the present invention may be Staphylococcus aureus, Listeria innocua, Listeria monocytogenes, Enterococcus faecium and/or Enterococcus faecalis. Exemplary gram-negative bacteria of the present invention may be Escherichia coli and Salmonella Typhimurium. The Escherichia coli of the present invention may comprise the O157:H7 serotype.

In an additional aspect, the present invention further concerns the use of at least one (one and/or more than one) lactic acid bacterium strain for reducing and/or treating food-borne pathogen infections. ‘Food-borne pathogen infections’ encompass, but are not limited to, listeriosis, salmonellosis, enterohemorragic Escherichia coli infections, staphyloenterotoxicosis, etc.

It is to be understood herein that by ‘reducing’ it is meant a process by which the infections (for example, food-borne and/or nosocomial) and/or microorganisms may be reduced, delayed, prevented and/or impaired.

It is also to be understood herein that by ‘treating’ it is meant a process by which the symptoms of infections (for example, food-borne and/or nosocomial infections) may not worsen, may remain stable, may be reduced and/or may be completely eliminated.

In an additional aspect thereof, the present invention also concerns the use of at least one lactic acid bacterium strain for reducing and/or treating nosocomial infections. Nosocomial infections are infections that may be caused by nosocomial pathogens. Nosocomial infections may be acquired incident to medical therapy.

A further aspect of the present invention provides for the use of at least one lactic acid bacterium strain for inhibiting (reducing, decreasing, lowering, impairing) the growth of spoilage microorganisms in a food product. Spoilage microorganisms may cause food to deteriorate. By inhibiting the growth of food spoilage microorganisms, the shelf-life of a food product may be increased. A ‘food product’ may be any product that has nutritive value and is suitable for ingestion into the gastrointestinal tract. The food products of the present invention may be fermentable or non-fermentable food products.

In an additional aspect, the present invention relates to a whey that may be obtained from fermentation using at least one lactic acid bacterium strain. By ‘whey’ it is meant a soluble fraction obtained after fermentation (for example, but without limitation, of milk and/or soy products), the fraction being substantially free of bacteria. For example, the whey may be obtained from the fermentation using at least one lactic acid bacterium strain after centrifugation or precipitation of insoluble solids. The whey may comprise, for example, a bacteriocin or bacteriocin-like substance.

In an exemplary embodiment of the invention, the whey may be obtained by fermenting probiotic bacteria in a fermentable food product using the following process:

Firstly, Lactobacillus acidophilus (comprising Lactobacillus I-1492) and Lactobacillus casei strains are incubated in a MRS type fermentation medium according to a standard program comprising several steps. The recombined lacteal base, which is partially lactose-free and degassed, is pasteurized for 1.5 minutes at 95° C. and inoculated at 10%. Finally, it is incubated according to the following program:

1) the I-1492 strain: 2 hours at 37° C.;

2) the acidophilus strain: 2 hours at 37° C. and

3) the casei strain: 1 hour at 37° C.

The product is then co-fermented (co-cultured) in an anaerobic atmosphere and medium for 15 hours at 37° C. (degassing under CO₂).

Although total amino acid content in such composition is similar to milk, free amino acids are significantly higher. The level of peptides comprised in the composition of the invention, having a molecular weight between 1000 and 5000 Da is around 30% and the level of small peptides having less than 10 residues is approximately 15%. It is known that such levels of peptides fortify the immune and digestive systems.

The present invention relates in yet a further aspect to the use of a whey for inhibiting (reducing, decreasing, lowering, impairing) the growth of microorganisms. The present invention also relates to the use of a whey for reducing and/or treating food-borne pathogen infections.

The present invention further relates to the use of a whey for inhibiting (reducing, decreasing, lowering, impairing) the growth of spoilage microorganism in food products.

In an exemplary embodiment of the present invention, the whey may have an acidic and/or neutral pH. In another exemplary embodiment, the whey may be irradiated. The whey may also be neutralized (neutral) and irradiated. The whey may also be acidic and irradiated.

In an additional aspect thereof, the present invention relates to a method for inhibiting the growth of microorganisms which may comprise the step of administering an effective amount of at least one lactic acid bacterium strain and a pharmaceutically and/or nutritionally acceptable vehicle.

An ‘effective amount’ is the necessary quantity to obtain positive results without causing excessively negative effects in the host to which the lactic acid bacterium strain may be administered. An effective amount is a quantity which may be sufficient to inhibit in any manner the growth of microorganisms.

An effective amount may be administered in one or more administrations, according to a regimen. The privileged method of administration and the quantity that may be administered is function of many factors. Among the factors that may influence this choice are: the condition, the age and the weight of the host to which the strain and/or whey is to be administered. In an exemplary embodiment, the administration in the present invention is oral administration. Oral administration may comprise any food forms and/or any food supplements including, but not limited to, capsules, tablets, liquid bacterial suspensions, dried oral supplements, wet oral supplements, dry tube feeding and/or wet tube feeding

By ‘pharmaceutically acceptable vehicle’ it is meant a vehicle that can be administered to a mammal, in particular to a human, with little or no negative or toxic side effects. Such a vehicle may be used for different functions. For example, it may be used as a preservation, solubilizing, stabilizing, emulsifying, softening, coloring, odoring and/or as an antioxidant agent.

By ‘nutritionally acceptable vehicle’ it is meant any liquid or solid form of nourishment that an organism (such as a mammal; in particular in a human) may assimilate.

In yet another aspect thereof, the present invention relates to a method for inhibiting the growth of microorganisms which may comprise the step of administering an effective amount of a whey obtained from fermentation using at least one lactic acid bacterium strain and/or a pharmaceutically and/or nutritionally acceptable vehicle as well as a method for reducing or treating food-borne pathogen infections, the method which may comprise the step of administering an effective amount of at least one lactic acid bacterium strain and/or an effective amount of a whey obtained from at least one lactic acid bacterium strain and/or a pharmaceutically and/or nutritionally acceptable vehicle.

The present invention further relates to a method for reducing and/or treating nosocomial infections. The method may comprise the step of administering an effective amount of at least one lactic acid bacterium strain and/or an effective amount of a whey obtained from at least one lactic acid bacterium strain and a pharmaceutically and/or nutritionally acceptable vehicle.

The present invention further relates to a method to inhibit the growth of spoilage microorganisms which may comprise the step of adding an effective amount of at least one lactic acid bacterium strain to a food product and/or which may comprise the step of adding an effective amount of a whey from fermentation using at least one lactic acid bacterium strain to a food product.

In yet a further aspect, the present invention concerns the use of at least one lactic acid bacterial strain and/or a whey obtained from fermentation using at least one lactic acid bacterium strain for modulating the gut flora. By ‘modulation’ it is meant to either increase and/or decrease the development of the gut flora (microbiota), whichever is advantageous to the host. By ‘gut flora’ it is meant the microbial flora which normally inhabits the human gut. Examples of gut flora are members of the Enterobacteriaceae, Bacteriodes, E. coli, Bifobacteria, Anaerobic cocci, Eubacteria, Costridia, lactobacilli, and/or yeasts. For example, modulation may involve decreasing, suppressing, attenuating, diminishing, and/or arresting the development of deleterious gut flora. In an exemplary embodiment of the present invention, the deleterious flora may comprise bacteria of the Staphylococcus and/or Enterobacteria genus. Modulation may also promote, increase, intensify and/or augment the development of beneficial flora. In an exemplary embodiment of the present invention, the beneficial flora may comprise Lactobacilli and/or lactic acid bacteria. The present invention also pertains to a method for modulating the gut flora which may comprise the step of administering an effective amount of at least one lactic acid bacterium strain and a pharmaceutically and/or nutritionally acceptable vehicle.

A further aspect of the present invention provides for a food product which may comprise a whey obtained from fermentation using at least one lactic acid bacterium strain.

The following examples illustrate potential applications of the invention and are not intended to limit its scope. Modifications and variations may be made therein without departing from the spirit and scope of the invention.

### Example I

### Delay of Pathogens Generation Time by Lactic Acid Bacteria Bacterial Strains

The probiotic strains Lactobacillus acidophilus and Lactobacillus casei (CL1285 mixture) were obtained from Bio-K+ International Inc. (Laval, QC, Canada). Escherichia coli ATCC 25922 and Staphylococcus aureus ATCC 29213 were obtained from the American Type Culture Collection (Rockville, Md., USA). Enterococcus faecalis LSPQ 2724 and Enterococcus faecium LSPQ 3550 were purchased from Laboratoire de Sante Publique du Québec (Step-Anne-de-Bellevue, QC, Canada). Escherichia coli serotype O157:H7 and Salmonella Typhimurium SL1344 were provided by INRS-Institut Armand-Frappier, Laval, QC, Canada.

Lactobacilli were propagated in Lactobacilli MRS broth (MRS; Difco Laboratories, Detroit, Mich., USA) at 35° C. for 24 h. All other bacteria were propagated in Brain-Heart Infusion broth (BHI; Difco Laboratories) at 35° C. for 24 h. Bacterial strains were stored at −80° C. in their respective media containing 100 g L-1 glycerol (Laboratoires MAT, Montreal, QC, Canada). Before each experiment, the bacterial content of one vial was thawed, transferred to 9 ml of their respective media and activated by two consecutive incubations of 24 h at 35° C. Thereafter, bacteria were washed twice in sterile saline (8.5 g L-1) after centrifugation at 4° C. for 10 min at 6000 g.

### Fermentation

Forty (40) ml of skimmed milk (100 g L-1), sterilized at 110° C. for 20 min was used as the fermentation substrate. Activated and washed CL1285 mixture was diluted in 12 ml of saline and 1 ml of this suspension was added to the milk in order to obtain an initial population of 10⁸ cfu mL-1. The milk was then inoculated with pathogenic bacteria (10⁴ cfu mL-1). Two groups were evaluated: 1) group containing pathogenic bacteria alone; 2) group containing a mixture of pathogens and the CL1285 mixture.

### Microbial Analysis

Samples of microbial suspension (1 ml) were taken at 0 h, 4 h, 8 h, 24 h and 48 h of fermentation in order to evaluate the concentration of each bacterium in milk. Bacterial enumeration was done using the pour plate method on selective media. LAB were screened on Lactobacilli MRS agar under anaerobic atmosphere; S. aureus were pour-plated on Baird-Parker Agar (Difco), E. coli and S. Typhimurium were enumerated on MacConkey (Difco) and Enterococcus agar (Difco) was used for enumeration of E. faecalis and E. faecium. L. innocua was plated on Palcam agar (Difco). All dishes were incubated at 35° C. from 24 h to 48 h and colonies were counted using Darkfield Quebec Colony Counter (American Optical, Scientific instrument division, Keene, Ohio, USA).

### Calculation of the Generation Time

The antimicrobial capacity (inhibitory activity) of a probiotic culture consisting in L. acidophilus and L. casei was evaluated on the growth of several pathogenic bacteria by calculation of the generation time during the exponential growth phase, in absence and in presence of lactobacilli strains. The generation time is derived from the division rate according to the following formulas (Prescott et al. 2003):

k=[(log Nt−log N0)/0.301×t]

g=1/k

where k is the division rate (h−1), g is the generation time (h), Nt is the microbial enumeration (CFU ml-1) after t=8 hours of fermentation and N0 is the microbial enumeration (CFU ml-1) after 4 h.

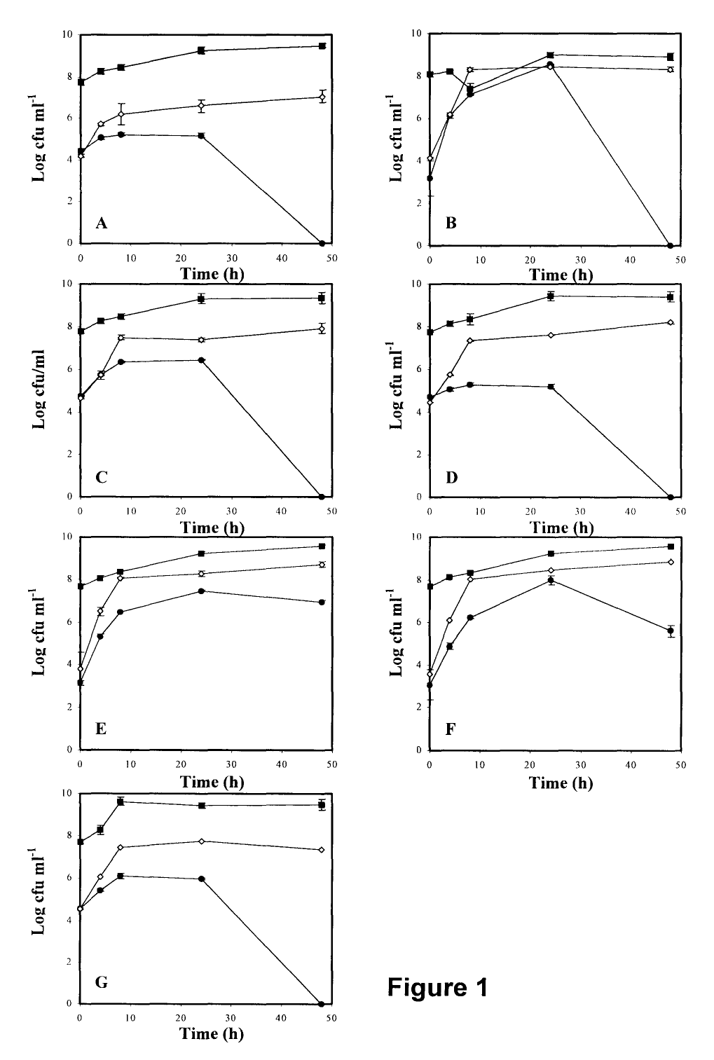

FIG. 1 (A-G) shows the growth of various pathogenic bacteria when cultured alone (monoculture) or in presence of the CL1285 culture (co-culture). The growth of all pathogens tested was inhibited. Indeed, most pathogens were completely eliminated in 48 h of fermentation in co-culture. Generation times of pathogenic bacteria in absence or in presence of CL1285 after 8 h of fermentation are presented in Table I.

TABLE I
GENERATION TIME OF PATHOGENS AFTER MONO- OR
CO-CULTURE WITH CL1285

| | Mono-culture | Co-culture | Generation |
|---|---|---|---|
| Pathogen | (min) | (min) | time increase |
| Enterococcus faecalis | 46.0 ± 0.2^{a} | 62.3 ± 6.3^{b} | ↑135% |
| LSPQ 2724 | | | |
| Enterococcus faecium | 37.8 ± 0.8^{a} | 53.1 ± 2.3^{b} | ↑140% |
| LSPQ 3550 | | | |
| Salmonella | 52.7 ± 0.9^{a} | 107.8 ± 5.3^{b} | ↑205% |
| Typhimurium | | | |
| SL1344 | | | |
| Escherichia coli | 34.4 ± 1.0^{a} | 71.5 ± 2.5^{b} | ↑208% |
| O157:H7 | | | |
| Listeria innocua | 41.8 ± 2.5^{a} | 99.0 ± 3.7^{b} | ↑237% |
| LSPQ 3285 | | | |
| Escherichia coli | 69.8 ± 16.4^{a1} | 174.0 ± 20.3^{b} | ↑249%² |
| ATCC 25922 | | | |
| Staphylococcus | 45.7 ± 0.6^{a} | 301 ± 13.5^{b} | ↑657% |
| aureus | | | |
| ATCC 29213 | | | |

¹Generation time in lines bearing different letters are significantly different (P ≦ 0.05)
²Increase of generation time (GT) = (Associative-culture GT/Mono-culture GT) × 100

LAB fermentation may result in a rapid acidification of the substrate. After 24 h of fermentation, the pH was reduced to 4.2 in the presence of CL1285. At this time, concentration of most of the pathogenic bacteria was reduced by 1 to 2 log₁₀, cfu mL-1. A discrete acidification of the milk to pH 3.9 was registered between 24 and 48 h of fermentation. During this period, a high bactericidal activity of 5 to 6 log₁₀, cfu mL-1 was observed, which caused the complete elimination of the pathogenic bacteria after 48 h of fermentation. Generation time of E. coli ATCC 25922 increased from 69.8 to 174.0 min when cultivated in presence of CL1285, representing an increase of 256%. Generation times of E. coli serotype O157:H7, L. innocua and S. Typhimurium increased from 34.4 to 71.5, from 41.8 to 99.0 and from 52.7 to 107.8 min, respectively. These results represented an increase of the generation time of more than 200%. S. aureus generation time increased from 45.7 to 301 min representing an increase of the generation time by 657% time. The presence of CL1285 increased the division time of E. faecium and E. faecalis from 37.8 to 53.1 and from 46 to 62.3 min, representing an increase of the generation time of 140 and 135% respectively. This observation suggests that antimicrobial metabolites other than organic acids are implicated in the elimination of the pathogenic bacteria. These results also show that after only 8 h of fermentation, LAB have the ability to increase the generation time of all the pathogenic bacteria evaluated in this study.

### Example II

### Delay of Pathogens Generation Time by Lactic Acid Bacteria Under Controlled pH

In order to eliminate the antimicrobial effect of the media acidification during fermentation by lactobacilli, mixed cultures were performed while maintaining the pH at 6.5 by a constant addition of KOH (5 mol l⁻¹). Each fermentation was conducted in a 1 L fermentor (BioFlo C30, New Brunswick Scientific Co., New Jersey, USA) equipped with pH and temperature probes. Fermentations were also conducted under agitation (250 rpm) for 48 h at pH 6.5 and 37° C. A volume (500 ml) of sterile reconstituted non-fat dry milk (10% w/v; RNDM; Difco) was inoculated with: 10⁴ CFU ml-1 pathogen or with a mixture of 10⁴ CFU ml-1 pathogen in presence of 10⁸ CFU ml-1 of probiotic culture. For each pathogen evaluated, two separate experiments were done. Samples (15 ml) were taken at 0 h, 4 h, 8 h, 24 h and 48 h in order to evaluate the concentration of each bacterium during fermentation. Bacterial enumeration was done using pour plate method on selective media.

Generation time of pathogenic bacteria in mixed cultures wherein pH was kept stable and neutral are presented in Table 2. The results show a clear increase of the generation time of Gram positive bacteria when grown in presence of the lactobacilli culture. Staph. aureus had a generation time of 40.5 min when cultured alone while the presence of the LAB increased the generation time to 84.8 min. L. innocua exhibited a generation time of 59.5 and 78.5 min under mono- and co-culture respectively. Ent. faecium and Ent. faecalis had a rapid growth when cultured alone showing a generation time of 39 min. However, in presence of lactobacilli culture, Ent. faecium was more sensitive than Ent. faecalis. Generation time of 62.7 and 43.2 min were respectively observed.

In contrast, Gram negative pathogenic bacteria were less sensitive to the presence of the lactobacilli culture under pH controlled condition. E. coli showed a decrease of the generation time in presence of the lactobacilli culture during mixed cultures with controlled pH. The generation time decreased from 93.1 to 56.4 min. Moreover, under these conditions, Salmonella Typhimurium was not affected by the presence of lactobacilli culture and showed a generation time of 58.2 min. Staphylococcus aureus was the most sensitive strain showing an increase of the generation time by 210%.

TABLE 2
GENERATION TIME OF PATHOGENS AFTER MONO- OR
CO-CULTURE WITH CL1285 UNDER STABLE pH CONDITIONS

| | Generation | Generation | Lactobacilli |
|---|---|---|---|
| | time in | time in | impact on |
| | monocultures | cocultures | generation time |
| Pathogen | (min) | (min) | (%) |
| Staphylococcus aureus | 40.5 ± 2.3^{a} | 84.8 ± 8.7^{b} | ↑ 210 |
| ATCC 29213 | | | |
| Enterococcus faecium | 39.5 ± 0.8^{a} | 62.7 ± 1.3^{b} | ↑ 160 |
| LSPQ 3550 | | | |
| Listeria innocua | 59.5 ± 4.4^{a} | 78.5 ± 4.2^{b} | ↑ 130 |
| LSPQ 3285 | | | |
| Enterococcus faecalis | 39.8 ± 0.6^{a} | 43.2 ± 2.2^{b} | ↑ 110 |
| LSPQ 2524 | | | |
| Salmonella Typhimurium | 58.2 ± 2.8^{a} | 58.5 ± 9.5^{a} | 0 |
| SL1344 | | | |
| Escherichia coli | 93.1 ± 6.1^{b} | 56.4 ± 6.0^{a} | ↓ 170 |
| O157:H7 EDL933 | | | |

^{a-b}Different letter means a significant difference between generation time of the mono- and co-culture P ≦ 0.05).

### Example III

### Whey Antimicrobial Activity

In order to verify the antimicrobial potential of the soluble fraction of fermented milk, 100 g of fermented milk was centrifuged at 16 500×g for 30 min at 4° C. and the supernatant was filter-sterilized (0.2 μm; Sarstedt, Montreal, QC, Canada). This supernatant was separated in three groups: “acidic fraction” (whey pH 4.5), “neutralized fraction” (whey pH 6.5) and “neutralized and irradiated fraction” (whey irradiated pH 6.5). The pH of the “acidic fraction” was 4.5. A portion of the supernatant was neutralized to pH 6.5 by addition of 5 mol l⁻¹ NaOH, in order to eliminate the antibacterial effect of the acidity against the pathogenic bacteria and this group was named “neutralized fraction”. Half of the neutralized fraction was irradiated at a dose of 45 kGy using a UC15-A irradiator in order to inactivate the possible antimicrobial peptides present in the supernatant. This group was named “irradiated and neutralized fraction”. 100 μl of BHI, 100 μl of treated supernatant, 50 μl of one of the pathogen suspension (approximately 10⁶ CFU ml-1) or sterile saline for the blank was added separately in flat bottom 96-wells plates (Sarstedt). The plates were then incubated at 35° C. and the microbial growth was monitored at 650 nm using a DMS-100S UV-Visible spectrophotometer every hour during 12 h until the bacterial stationary phase was reached.

The pathogen growth inhibition by the fermented milk soluble fraction (whey) was calculated after 12 h of incubation following the equation:

Aₛₐₘₚₗₑₛ−A_{blank}=A_{calculated}
Aₛₐₘₚₗₑₛ: absorbance of the well containing 50 μl of pathogenic bacteria in saline supension, 100 μl of fermented milk supernatant fraction and 100 μl of BHI.
A_{blank}: absorbance of the well containing the blank (50 μl saline), 100 μl of fermented milk supernatant fraction and 100 μl of BHI.
A_{calculated}: Calculated absorbance of each sample.

The percentage of pathogen inhibition was calculated as follows:

$$\frac{100 - {A}_{treatment}}{{A}_{BHI}} \times 100 = Inhibition \%$$

Aₜᵣₑₐₜₘₑₙₜ: Acalculated in presence of the pathogens in BHI and supernatant fraction.
A_{BHI}: Acalculated in presence of pathogens in BHI.
Inhibition %: percentage of growth inhibition of the pathogen by the presence of the fermented milk soluble fraction (whey).

Table 3 presents the inhibition percentage of selected pathogenic bacteria when cultivated in presence of the whey (soluble fraction) of L. acidophilus and L. casei-fermented milk.

TABLE 3
INHIBITION LEVEL (%) AFTER INCUBATION IN BHI
CONTAINING LACTIC ACID BACTERIA WHEY

| | Growth Inhibition % | | | | |
|---|---|---|---|---|---|
| | | | | | Whey |
| | | | | | irradiated |
| | | BHI | Whey | Whey | 45 kGy |
| | | pH 4.5 | pH 4.5 | pH 6.5 | pH 6.5 |
| Pathogens | BHI | (%) | (%) | (%) | (%) |
| Escherichia coli | 0 | 27.8 ^{a} ± 3.1 | 77.2 ^{c} ± 1.0 | 61.9 ^{b} ± 2.1 | 32.0 ^{a} ± 4.9 |
| O157:H7 | | | | | |
| Enterococcus | 0 | 23.9 ^{b} ± 5.8 | 75.3 ^{d} ± 1.1 | 52.6 ^{c} ± 3.3 | 39.1 ^{a} ± 4.2 |
| faecalis | | | | | |
| Enterococcus | 0 | 6.4 ^{a} ± 3.9 | 73.5 ^{d} ± 5.2 | 48.9 ^{c} ± 4.7 | 12.5 ^{b} ± 2.5 |
| faecium | | | | | |
| Listeria innocua | 0 | 41.1 ^{b} ± 4.4 | 85.9 ^{d} ± 3.7 | 65.9 ^{c} ± 2.1 | 25.9 ^{a} ± 2.7 |
| Listeria | 0 | 25.8 ^{b} ± 1.8 | 78.4 ^{d} ± 2.5 | 59.7 ^{c} ± 3.2 | 14.1 ^{a} ± 1.5 |
| monocytogenes | | | | | |
| Staphylococcus | 0 | 17.2 ^{a} ± 2.7 | 84.7 ^{d} ± 3.6 | 49.5 ^{c} ± 3.6 | 31.2 ^{b} ± 5.4 |
| aureus | | | | | |

^{a-e} Different letter means a significant difference between treatment (P ≦ 0.05).

The whey obtained from L. acidophilus and L. casei-fermented milk had a pH of 4.5. Pathogens growth inhibition evaluated in presence of acidic whey varied from 73.5% for Ent. faecium up to 85.9% for L. innocua after 12 h of incubation at 35° C. The bacterial sensitivity, in decreasing order, is: L. innocua (86%)>Staph. aureus (85%)>L. monocytogenes (78%)>E. coli O157:H7 (77%)>Ent. faecalis (75%)>Ent. faecium (74%).

In order to verify the antimicrobial role played by organic acids, neutralization of the soluble fraction to pH 6.5 was achieved. Such neutralization significantly reduced the inhibitory potential against all selected pathogens (P≦0.05). However, residual antimicrobial activity in neutralized soluble fraction was still observed suggesting the presence of antimicrobial compounds other than organic acids and/or bacteriocins.

pH neutralization and irradiation of L. acidophilus and L. casei-fermented milk supernatant was performed. The dose of irradiation utilized is normally used to inactivate enzymes. Growth inhibition results of neutralized and irradiated fraction on selected pathogens are presented by decreasing order of sensitivity: Ent. faecalis (39%)>E. coli O157:H7 (32%)>Staph. aureus (31%)>L. innocua (26%)>L. monocytogenes (14%)>Ent. faecium (13%).

L. innocua and Staph. aureus were the most sensitive bacteria to the presence of whey showing an inhibition of 85.9 and 84.7%, respectively. The most sensitive strains to neutralized whey were L. innocua and E. coli serotype O157:H7 showing an inhibition of 65.9% and 61.9%, respectively. Ent. faecalis, E. coli O157:H7 and Staph. aureus were the most affected bacteria by the neutralized and irradiated fraction showing 39.1%, 32% and 31.2% inhibition. These results suggest the implication of both organic acids and bacteriocin-like inhibitory substance in the antimicrobial activity observed in the whey of the probiotic preparation.

### Example IV

### Lab Survival of Gastrointestinal Transit

A persistent concern in the field of probiotics is the ability of probiotic strains to resist, survive and colonize the intestine at least temporarily. Viability and survival of probiotic bacteria are important characteristics in order to provide health benefits. Probiotic should survive the gastro-intestinal transit to colonize the gut. Natural resistance to gastro-intestinal transit varies between LAB species (Charteris et al. 1998). Indeed, certain strains have the capacity to resist more easily under the extreme acidity of stomach or to the bile salts in the small intestine (Grill et al.; Truelstrup et al.) The stomach pH depends on its content and can vary from about 1.5 to 3. It is estimated that only 20-40% of probiotic effectively survive the gastro-intestinal transit (Bezkorovainy et al., 2002).

### Lab Tolerance to Bile Salts

The bile salt tolerance of CL1285 (LAB) was ascertained in MRS agar containing a commercial preparation of bile salts (Sigma B-3426, Oakville, ON, Canada). Bile salts mixture was added in concentration varying between 0 and 100 g L-1. Bile salts containing-MRS agar was then autoclaved for 15 min at 121° C., cooled and finally plated. Overnight MRS broth cultures (100 μl of bacteria in the stationary phase of growth) were inoculated on surface of bile salts-containing MRS agar and incubated at 37° C. for 72 h under anaerobic conditions. Presence of a bacterial lawn indicated a good growth and thus good resistance of bacteria to bile salts while presence of small and isolated colonies or no colonies indicated a poor resistance to bile salts. Minimal inhibitory concentration represents the lowest concentration of the bile salts assayed which totally inhibited the growth of colonies as judged from visual examination. The minimal inhibitory concentration of the bile salts mixture for CL1285 was 50 g L-1 showing that CL1285 is resistant to bile salts.

### Lab Tolerance to Acid

Furthermore, the acid tolerance of CL1285 (LAB) was using simulated gastric fluid (SGF) formulated according to United States Pharmacopae (USP). SGF was composed of 3.2 g L-1 of pepsin (Sigma), 2.0 g L-1 NaCl and pH was finally adjusted to 1.5, 2.0, 2.5 or 3.0 by addition of HCl (5 mol L-1). A volume of one ml of overnight MRS broth cultures of LAB were added to 19 ml of SGF for 30 min at 37° C. under mild agitation (200 rpm) in a G24 Environmental Incubator Shaker (New Brunswick Scientific Co. Inc., NJ, USA). After 30 min in gastric solution, 1 ml was collected and mixed in sterile PBS (pH 7.4). A similar process was carried out for bacteria without SGF treatment in order to determine the initial concentration of LAB. To perform viable cell determination, appropriate dilutions from these samples were done in sterile peptone water (10 g L-1) and plated on Lactobacilli MRS agar. Plates were incubated under anaerobic conditions at 35° C. for 48 h. The average number of colony-forming units (cfu) from triplicate analysis was determined by Darkfield Quebec Colony Counter. Results presented in Table 4 show that tested bacteria can survive (resist, tolerate) an acidic environment during at least 30 min. These results show that the CL1285 probiotic preparation completely resists a simulated gastric fluid at pH≧2.5.

TABLE 4
SURVIVAL OF LACTIC ACID BACTERIA STRAINS
AFTER INCUBATION IN SIMULATED GASTRIC FLUID

| LAB Strains | Time (min) | pH | Log CFU survivor |
|---|---|---|---|
| L. acidophilus/L. casei CL1285 | 0 | — | 9.57 ± 0.09^{B} |
| | 30 | 1.5 | <1 |
| | | 2.0 | 5.90 ± 0.52^{A} |
| | | 2.5 | 9.55 ± 0.04^{B} |
| | | 3.0 | 9.63 ± 0.04^{B} |

*: Different letters in a bacterial group indicate a significant difference in microbial count (P ≦ 0.05)

### Example V

### Lab Modulation of the Fecal Microbiota

Survival in the stressful gastrointestinal transit may allow probiotics to reach the gut. The presence of live probiotic in the gut could be beneficial to humans by establishing an healthy gut flora and by preventing invasion by deleterious bacteria. The composition of gut flora could be altered specifically following ingestion of probiotics. The impact of LAB on the gut flora measured via fecal microbiota quantitation was tested in vivo.

Six- to eight-week-old female C57BL/6 mice were housed in plastic cages and kept under pathogen-free conditions with free access to commercial chow and water. Healthy mice received a daily dose of about 10⁹ viable bacteria (CL1285 mixture) in 100 μl of PBS by intragastric route using a stainless steel feeding needle and a 1 ml syringe. Mice were weighed at day 1, 9, 18, and then 9 days after the end of the feeding treatment (day 27-post feeding). Stool samples were collected before the administration of PBS or probiotics at day 1, 9 and 18 after the beginning of the feeding procedures. Final analysis was done 9 days after the end of the treatment (day 27-post feeding).

### Quantification of Stool Organisms

Fresh stool samples were weighed, diluted in 1000 μl of sterile saline, homogenized with a pestle, 10-fold serially diluted in 10 g L-1 peptone water and finally 100 μl were inoculated on the following selective media: Lactobacilli MRS agar for detection of total lactic acid bacteria (LAB), Rogosa SL agar for detection of Lactobacilli sp., Reinforced Clostridium Medium (RCM) for quantification of total anaerobic mesophilic bacteria. Baird-Parker agar (BPA) for detection of Staphylococci sp. and MacConkey agar for enumeration of Enterobacteriaceae. MRS, Rogosa and RCM plates were incubated in anaerobic jars at 37° C. for 72 h while BPA and MacConkey plates were incubated under aerobic conditions at 37° C. for 48 h.

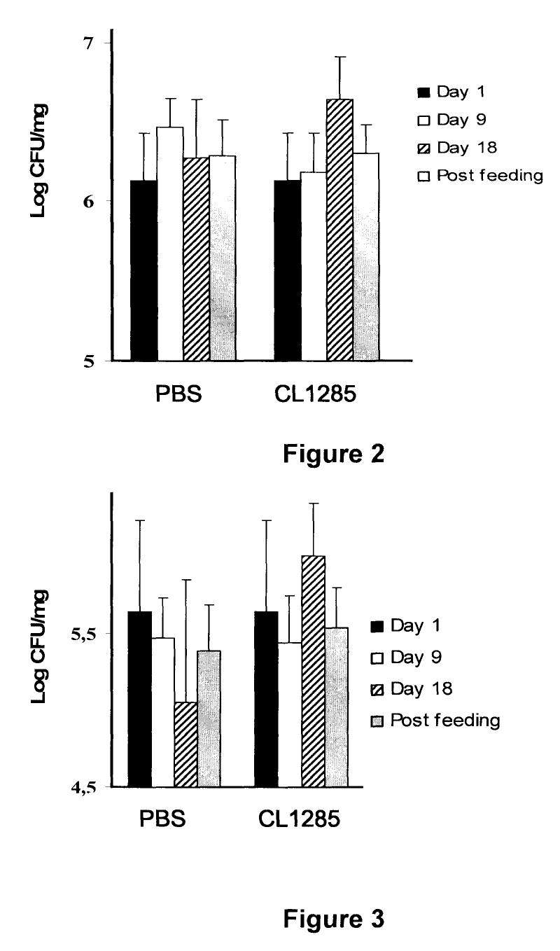

The composition of microbial populations in C57BI/6 mice fecal samples is shown in FIGS. 2 to 6. The LAB counts in the mice feces were significantly higher after 18 days of CL1285 ingestion than after PBS ingestion (P≦0.05). However, after feeding ended, the level of LAB was similar to its initial count.

Lactobacillus sp. population were not greatly affected (P>0.05) by the bacterial composition of all probiotics evaluated (FIG. 3).

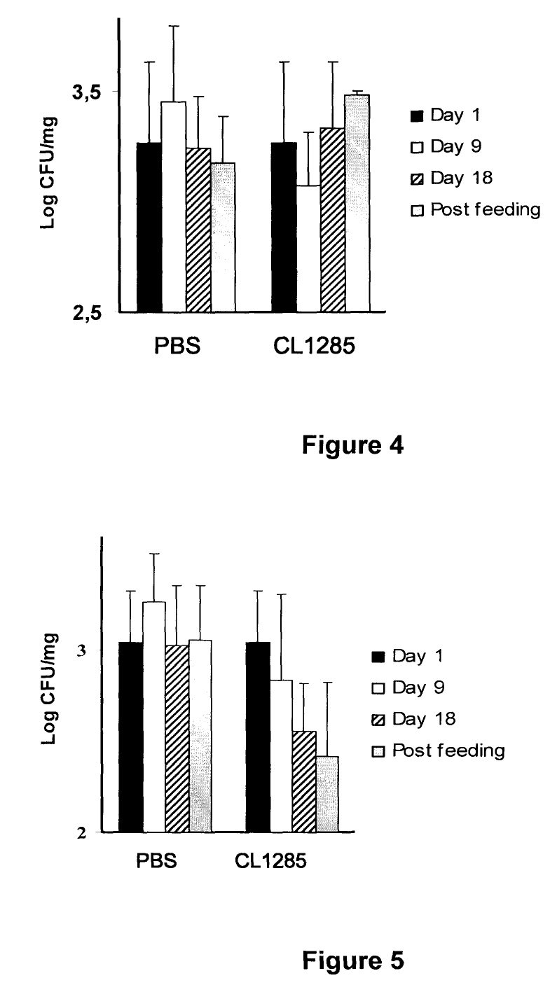

Fecal Enterobacteriaceae counts were affected at day 9 by the presence of CL1285. This reduction was temporary as seen by the increase in Enterobacteriaceae population observed after feeding with CL1285 was stopped (FIG. 4).

The consumption of CL1285 led to a significant reduction in Staphylococci population (P≦0.05) at all days assayed even post-feeding (FIG. 5).

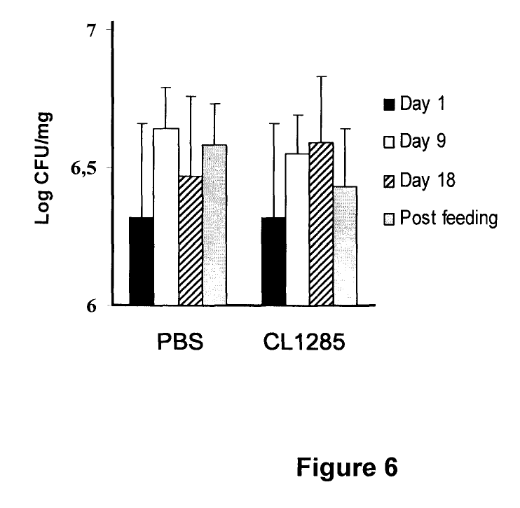

Finally, FIG. 6 shows the total anaerobe counts in feces. An increase of these populations was observed. This increase was observed until the end of the treatment and post-feeding.

These results also demonstrate that ingestion of CL1285 probiotics is well tolerated by C57BI/6 mice over the course of a three week-feeding trial and can alter quantitatively the balance of colonic bacterial populations.

All the probiotics tested have the potential to increase, at least transiently, the total culturable LAB content. Although the mice were fed routinely with probiotic Lactobacillus strains, the increase in total LAB was not correlated with an elevation of the Lactobacilli population. A plausible reorganization of the gut flora might have been induced and the probiotic species replaced or stimulated the growth of the indigenous Lactobacillus strains leading to a variation of the bacterial species but not to a quantitative modification. The same hypothesis could explain the increase in total anaerobes.

Bacterial populations of Enterobacteriacea and Staphylococci that are often considered deleterious microorganisms were also studied. The population of total LAB in the feces increased while Staphylococci decreased following the feeding of mice. A constant reduction of Staphylococci was noticed following the ingestion of CL1285.

Although the present invention has been described by way of exemplary embodiments, it should be understood by those skilled in the art that the foregoing and various other changes, omission and additions may be made therein and thereto, without departing from the spirit and scope of the present invention.

### REFERENCES

Adolfsson, O., Meydani, S. N. and Russell, R. M. (2004). Yogurt and gut function. Am J Clin Nutr 80, 245-56.
Bezkorovainy A., Probiotics: determinants of survival and growth in the gut, Am. J. Clin. Nutr. 73 supp. 2 (2002) 399S-405S.
Charteris W. P., Kelly P. M., Morelli L., Collins J. K., Development and application of an in vitro methodology to determine the transit tolerance of potentially probiotic Lactobacillus and Bifidobacterium species in the upper human gastrointestinal tract. J. Appl. Microbiol. 84 (1998) 759-768.
De Buyser M. L., Dufour B., Maire M., Lafarge V., Implication of milk and milk products in food-borne diseases in France and in different industrialised countries, Int. J. Food Microbiol. 67 (2001) 1-17.
Ghrairi, T., Manai, M., Berjeaud J. M. and Frere, J. (2004). Antilisterial activity of lactic acid bacteria isolated from rigouta, a traditional Tunisian cheese. J Appl Microbiol 97, 621-628.
Grill J. P., Manginot-Dürr C., Schneider F., Ballongue J., Bifidobacteria and probiotic effects: action of Bifidobacterium species on conjugated bile salts, Current Microbiol. 31 (1995) 23-27.
Gusils, C., Oppezzo O., Pizarro R., and Gonzalez, S. (2003) Adhesion of probiotic Lactobacilli to chick intestinal mucus. Can. J. Microbiol. 49:472-478.
Naaber, P., Smidt, I., Stsepetova, J., Brilene, T., Annuk, H. and Mikelsaar, M. (2004). Inhibition of Clostridium difficile strains by intestinal Lactobacillus species. J Med Microbiol 53, 551-554.
Nes, I. F. and Johnsborg, O. (2004). Exploration of antimicrobial potential in LAB by genomics. Curr Opin Biotechnol 15, 100-104.
Perdigon, G., Locascio, M., Medici, M., Pesce de Ruiz Holgado, A. and Oliver, G. (2003). Interaction of Bifidobacteria with the gut and their influence in the immune function. Biocell 27, 1-9.
Rossland, E., Andersen Borge, G. I., Langsrud, T. and Sorhaug, T. (2003). Inhibition of Bacillus cereus by strains of Lactobacillus and Lactococcus in milk. Int J Food Microbiol 89, 205-212.
Scheinbach S. (1998). Probiotics: functionality and commercial status. Biotechnol Adv 16, 581-608.
Truelstrup Hansen L., Allan-Wojtas P. M., Jin Y. L., Paulson A. T., Survival of Ca-alginate microencapsulated Bifidobacterium spp. in milk and simulated gastrointestinal conditions, Food Microbiol. 19 (2002) 35-45.

1. A method for inhibiting the growth of food-borne pathogens, nosocomial pathogens and spoilage microorganisms, the method comprising contacting at least one lactic acid bacterium strain selected from the group consisting of Lactobacillus acidophilus I-1492, Lactobacillus casei and mixture thereof or a whey obtained from fermentation of said lactic acid bacterium strain with the microorganisms.

2-7. (canceled)

8. The method of claim 1, wherein the microorganisms are selected from the group consisting of gram-positive bacteria and gram-negative bacteria.

9. The method of claim 8, wherein the gram-positive bacteria are selected from the group consisting of Staphylococcus aureus, Listeria innocua, Listeria monocytogenes, Enterococcus faecium and Enterococcus faecalis.

10. The method of claim 8, wherein the gram-negative bacteria are selected from the group consisting of Escherichia coli and Salmonella Typhimurium.

11. The method of claim 10, wherein the Escherichia coli comprises the O157:H7 serotype.

12. A method for reducing or treating food-borne pathogen infections, the method comprising administering at least one lactic acid bacterium strain selected from the group consisting of Lactobacillus acidophilus I-1492, Lactobacillus casei and mixture thereof or a whey obtained from fermentation of said lactic acid bacterium strain to a mammal in need.

13-14. (canceled)

15. A whey obtained from fermentation of Lactobacillus acidophilus I-1492.

16-18. (canceled)

19. The whey according to claim 15, wherein the whey has an acidic or neutral pH.

20. The whey according to claim 15, wherein the whey is irradiated.

21. A method for modulating a gut flora, the method comprising administering at least one lactic acid bacterium strain selected from the group consisting of Lactobacillus acidophilus I-1492, Lactobacillus casei and mixture thereof to a mammal in need.

22-24. (canceled)

25. The method of claim 21, wherein the modulation involves inhibiting the development of deleterious flora.

26. The method of claim 25, wherein the deleterious flora comprises bacteria of the Staphylococcus and Enterobacteria genus.

27. The method of claim 25, wherein the deleterious flora consist of bacteria of the Staphylococcus genus.

28. The method of claim 21, wherein the modulation involves promoting the development of beneficial flora.

29. The method of claim 28, wherein the beneficial flora comprises lactobacilli and lactic acid bacteria.

30. A food product comprising a whey obtained from fermentation of at least one lactic acid bacterium strain selected from the group consisting of Lactobacillus acidophilus I-1492, Lactobacillus casei and mixture thereof.

31-33. (canceled)
